(19) United States
(12) Patent Application Publication
(10) Pub. No.: US 2016/0201112 A1
(43) Pub. Date: Jul. 14, 2016

(54) NANOSENSOR FOR DETECTING THE ACTIVITY OF GLYCOSAMINOGLYCAN-CLEAVING ENZYMES AND USES THEREOF

(71) Applicant: VIRGINIA COMMONWEALTH UNIVERSITY, Richmond, VA (US)

(72) Inventors: Umesh R. Desai, Glen Allen, VA (US); Mausam Kalita, Salt Lake City, UT (US); Kuberan Balagurunathan, Salt Lake City, UT (US)

(73) Assignee: VIRGINIA COMMONWEALTH UNIVERSITY, Richmond, VA (US)

(21) Appl. No.: 14/912,408

(22) PCT Filed: Sep. 8, 2014
(86) PCT No.: PCT/US2014/054490

§ 371 Date: Feb. 17, 2016

Related U.S. Application Data
(60) Provisional application No. 61/875,427, filed on Sep. 9, 2013.

(51) Int. Cl. C12Q 1/527; G01N 21/64; G01N 33/58; C08B 37/00

(52) U.S. Cl. CPC C12Q 1/527; C08B 37/0075; G01N 21/6428; G01N 33/582; G01N 2333/988; G01N 2400/40; G01N 2021/6432; G01N 2021/6439

(57) ABSTRACT

High sensitivity nanosensors for detecting the inhibition of glycosaminoglycan (GAG)-cleaving enzymes are provided. Methods of using the nanosensors include detecting contaminants in commercial GAG preparations (e.g. heparin preparations) by measuring the activity levels of a GAG)-cleaving enzyme in the presence of a sample which may contain a contaminant that inhibits the GAG-cleaving enzyme.

### BACKGROUND OF THE INVENTION

### Field of the Invention

The invention generally relates to methods of detecting the activity levels of glycosaminoglycan (GAG)-cleaving enzymes. In particular, the invention provides nanosensors with very high sensitivity for detecting the inhibition of glycosaminoglycan-cleaving enzymes, for example, inhibition by contaminants in commercial preparations of glycosaminoglycans such as heparin.

### BACKGROUND OF THE INVENTION

Heparin is a highly sulfated natural polysaccharide used as an anticoagulant agent in various medical procedures such as deep vein thrombosis and acute coronary syndromes.¹⁻³ Heparin consists of sulfated disaccharide repeating unit of iduronic acid (or glucoronic acid) and glucosamine.^{4,5} However, due to its animal origin, heparin lots can be contaminated with other sulfated glycoseaminoglycans (GAGs). In addition, economically motivated adulteration with non-heparin components is also a troubling source of contamination.

In 2007-2008, adulteration in the global heparin supply chain led to severe adverse reactions in patients such as angiodema, hypotension, and swelling of the larynx.^{6,7} Further, kidney dialysis patients suffered from an anaphylactoid reaction (AR).⁸ Synthetic oversulfated chondroitin sulfate (OSCS), an unnatural highly sulfated polysaccharide was identified as the principal contaminant in these cases.⁶ This economically motivated heparin contamination resulted in 574 reports of adverse effects and 94 deaths in the United States and European nations.^{6,7,9} In addition to being used for administration to patients, heparin is also used in more than 200 medical devices and diagnostic tool kits to reduce coagulation during use of the device or diagnostic.¹⁰ Therefore, maintaining a high quality heparin supply chain is absolutely critical to reduce adverse bleeding effects in patients and to safeguard the excellence of medical devices in the market.

In order to detect OSCS contamination in heparin, several analytical methods have been developed. These analytical techniques include 1D-¹H NMR,¹¹ strong anion exchange (SAX)-HPLC,¹² capillary electrophoresis (CE),¹³ polyacrylamide gel electrophoresis (PAGE),¹⁴ near-infrared (NIR)¹⁵ and electrochemical methods.¹⁶ The limit of detection (LOD) varies from 1% (NIR) to 0.03% (SAX-HPLC) w/w OSCS in heparin.¹¹⁻¹⁶ While these techniques can accurately detect OSCS and other oversulfated impurities in heparin, they have low detection limits and also disadvantageously require the use of sophisticated instrumentation and data analysis.

To overcome these challenges, researchers have also developed 96-well microplate assays to detect OSCS contaminant. These assays take advantage of probes that undergo colorimetric, fluorescent and/or enzymatic transformations upon treatment with OSCS spiked heparin.¹⁷⁻¹⁹ The most sensitive colorimetric assay to date has a limit of detection (LOD) of 0.003% w/w of OSCS in heparin.¹⁷ This method employs water soluble cationic Leclerc Poly Thiophene Polymer (LPTP), a yellow colored probe that interacts with polyanionic heparin, resulting in a color change from yellow to red.²⁰ However, the yellow color remains unchanged when heparin is contaminated with 1-10% w/w of OSCS, and LPTP cannot distinguish between 0.001% and 0% w/w OSCS contamination.¹⁷ Thus, to detect lower contamination levels, it is necessary to use other or additional detection methods.

There is an urgent, unmet need in the art to detect heparin contaminants at very low levels, using methods and/or devices that are rapid, cost-effective, and which do not require excessively sophisticated instrumentation.

Kwon et al. (U.S. Pat. No. 8,551,727 B2, the complete contents of which is hereby incorporated by reference in entirety) teaches a nanoprobe designed to detect the presence of proteases expressed in cells or tissue, including detection in vivo. The nanoprobe comprises a fluorophore linked to a peptide that has a unique degradability with respect to a protease of interest. The only peptide sequence provided by Kwon is found in example 1 (column 6), which teaches that the peptide with an ester linkage to a Cy5.5 fluorophore (Cy5.5-GPLGLFARC) and is specific for detection of a protease known as matrix metalloproteinase-2 (MMP-2). The gold nanoparticle is prepared by reducing gold salt using sodium borohydride and chemically coupling the reduced gold to Cy5.5-GPLGLFARC. However, the invention of Kwon encompasses only nanoprobes which comprise peptides, and methods of detecting proteases therewith.

### SUMMARY OF THE INVENTION

Contaminants in heparin preparations are highly toxic, and the detection of low levels of contaminants is a heretofore unresolved problem in the industry. Embodiments of the present invention provide a solution to this problem by providing nanoprobes and assay systems using the nanoprobes that detect contaminants at very low (e.g. femtomolar) concentrations. The nanoprobes are designed to detect the activity level of an enzyme that is capable of cleaving a GAG of interest such as heparin. When the enzyme is incubated with a sample of interest that contains a contaminant that inhibits the enzyme, the level of activity of the enzyme is lower than if no contaminant/inhibitor is present in the sample. In an exemplary aspect, the GAG is heparin and the inhibitor that is detected is the contaminant OSCS. The nanoprobe of the invention is extremely sensitive, detecting inhibitors that are present at femtomolar concentrations. The nanoprobe is easy to synthesize, prepare, or supply in usable form. Assays using the nanoprobe are rapid (results are obtained within about 30 minutes) and convenient (tests can be carried out using e.g. standard 96-well microplates). The nanoprobe is well-suited for use in high-throughput screening and diagnostic assays.

In an exemplary aspect, the GAG in the nanoprobe is heparin, the signaling molecule is a fluorescent dye, and the metal is a metal that is capable of quenching the fluorescence of the dye, e.g. gold. In this aspect, the enzyme activity that is assessed is heparin-cleavage, and the presence or absence of an enzyme inhibitor such as the contaminant OSCS is detected. Exemplary applications of the nanosensors include: the detection, in a sample, of substances that inhibit the activity of a GAG-cleaving enzyme of interest; the measurement of the activity level of a GAG-cleaving enzymes of interest, e.g. measurement of batch to batch variations of manufactured enzyme preparations; and various industrial and forensic applications.

It is an object of this invention to use nanoprobes to assess an amount or activity of a glycosaminoglycan (GAG)-cleaving enzyme in a sample of interest. The methods described herein have many applications. For example, the method is used to determine enzyme kinetics for the GAG-cleaving enzyme (e.g. Kₘ of binding to the GAG). In some embodiments, the invention is used to detect at least one sulfated polysaccharide contaminant in a heparin preparation or heparin-containing solution. In other embodiments, the invention is used to assess an amount or therapeutic activity of a protein or glycan present in a patient in need thereof.

Other features and advantages of the present invention will be set forth in the description of invention that follows, and in part will be apparent from the description or may be learned by practice of the invention. The invention will be realized and attained by the compositions and methods particularly pointed out in the written description and claims hereof.

### BRIEF DESCRIPTION OF THE DRAWINGS

FIGS. 1A and B. TEM images of A, Au-Heparin-Dye nanosensor and B, Heparitinase treated Au-Heparin-Dye nanoparticle after 4 h of incubation. The average size of the particles is ˜18 nm in both cases.

FIG. 2A-C. A, Au-Heparin-Dye nanosensor shows ˜88% reduction in fluorescence due to NSET. B, Incubation of the nano-probe with a heparitinase enzyme results in a gradual increase in fluorescence intensity. An enhancement of ˜70% fluorescence intensity is recorded over a period of 4 h. C, heat map of fluorescence increase is captured through charge coupled device (CCD) digital camera using a DS red filter (575 nm-656 nm).

FIG. 3A-C. HPLC profile of heparin disaccharides after incubating heparitinase enzyme with A) 100% heparin. B) 90% heparin +10% w/w CS A and C. C) 90% heparin +10% w/w OSCS. In these concentrations, 10% w/w OSCS completely inhibits the enzyme.

FIGS. 4A and B. A) The images of the 96-well plate are captured after exciting the wells with 535 nm and recording the emission with DS red filter (575 nm-656 nm). The images are recorded at −5 min (before addition of heparitinase), 1 min, 30 min, 1 h, 2 h, 3 h and 4 h after incubating the nano-probe with the heparitinase enzyme@ heparin-OSCS. B) A plot of relative photon count of the wells Vs [OSCS] w/w % in heparin shows that the CCD camera, effectively detects 0.1 ppm OSCS contaminant in heparin. This plot strongly affirms the ultra-sensitivity of the nanosensor.

FIG. 5. Schematic depiction of assays using the nanoprobe of the invention.

### DETAILED DESCRIPTION

Embodiments of the invention include a nanosensor for detecting contamination in samples of interest. The nanosensor is capable of sensing the level of activity of glycosaminoglycan (GAG)-cleaving enzymes, e.g. hydrolases, eliminases, etc. The nanosensor comprises a substrate of a GAG-cleaving enzyme of interest. The substrate is chemically attached to both 1) at least one signaling molecule that produces a detectable signal when free (e.g. one or more fluorescent dye molecules in an aqueous or non-aqueous solution); and 2) a metal nanoparticle that quenches (prevents or lessens) the detectable signal by the signaling molecule when the signaling molecule is attached to the nanosensor. When at least one nanosensor is present in a reaction mixture together with at least one GAG-cleaving enzyme of interest, the enzyme cleaves the GAG and releases the signaling molecule from the nanoprobe (e.g., into solution or other environment where detection is possible). This results in production of a detectable signal by the freed signaling molecule. However, if cleavage of the GAG is prevented, e.g. by inhibition of the GAG-cleaving enzyme, then the signaling molecule is not released, or fewer of the signaling molecules are released, and the amount of detectable signal decreases accordingly. Therefore, a lowered or attenuated level of fluorescence intensity indicates that an enzyme inhibitor is present in the sample.

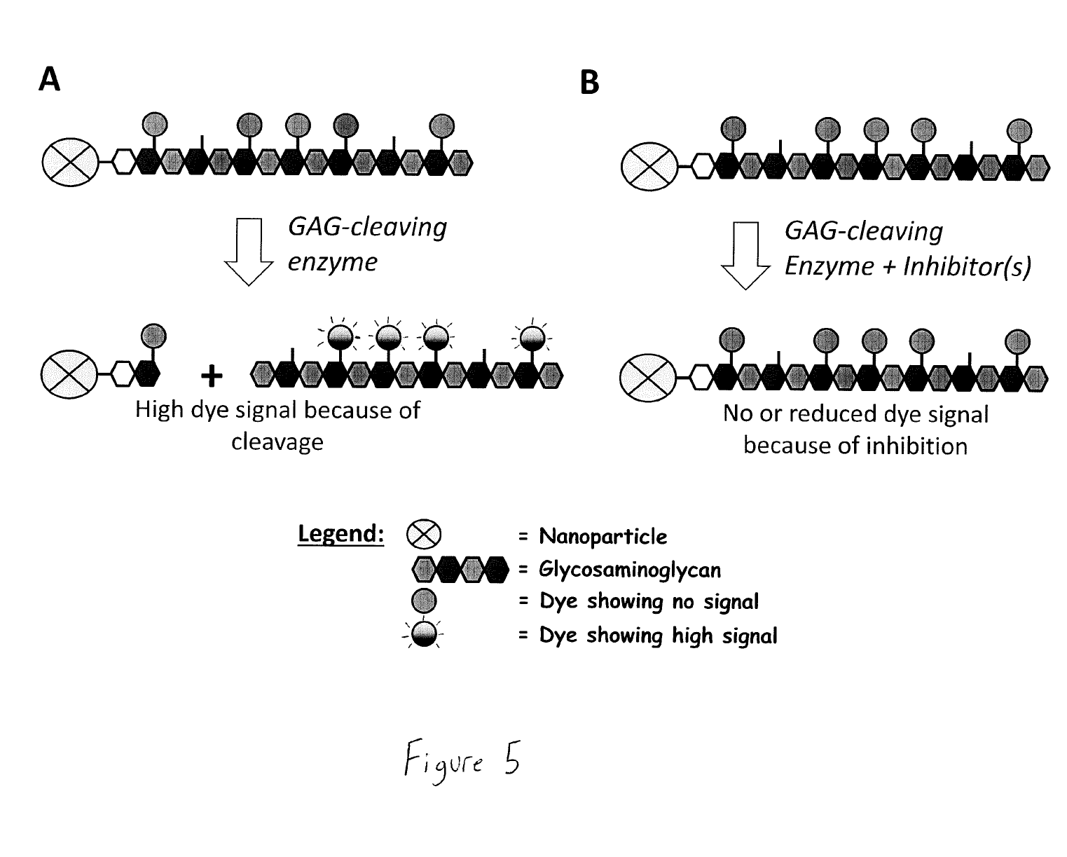

FIG. 5 depicts a schematic of the use of the nanoprobe. The nanoprobe is shown as comprising signaling molecules (fluorescent dye) attached to GAG substrate, which is in turn tethered to a nanoparticle. Pathway A shows the results when the nanoprobe is combined in solution with an enzyme capable of cleaving GAG substrate in the absence of a contaminant/inhibitor. As can be seen, the enzyme cleaves the GAG and signaling molecules are released and produce a detectable signal. Pathway B shows the results when nanoprobe is combined in solution with an enzyme capable of cleaving GAG substrate and when there is also a contaminant/inhibitor of the enzyme in the solution. The enzyme is inhibited, the GAG is not cleaved, the signaling molecule remains attached to the nanoprobe, and no detectable signal is produced. Those of skill in the art will recognize that in this schematic, extremes of the reactions are depicted for simplicity. For example, in solution, many nanoprobes are present and all may not be cleaved, even if no inhibitor is present. Further, even when an inhibitor is present, all enzyme cleavage may not be prevented, i.e. some signal may be detectable. However, in general, the amount of signal is attenuated when an enzyme inhibitor is present in the solution. Further, in the assays of the invention, the amounts of nanoprobe and enzyme are calibrated as needed to maximize the contrast between samples with inhibitors and without inhibitors, as compared to suitable controls.

### Definitions

The following definitions are used throughout:

Fluorescence quenching is the loss of fluorescence intensity which is observed when a fluorescent molecule or group interacts with another molecule or group, called the quencher. Heparin: Native heparin is a polymer with a molecular weight ranging from 3 to 30 kDa, the average molecular weight of most commercial heparin preparations being in the range of 12 to 15 kDa. Heparin is a member of the glycosaminoglycan family of carbohydrates (which includes the closely related molecule heparan sulfate) and consists of a variably sulfated repeating disaccharide units. The most common disaccharide unit is composed of 2-O-sulfated iduronic acid and 6-O-sulfated, N-sulfated glucosamine, IdoA(2S)-GlcNS(6S). Disaccharides containing a 3-O-sulfated glucosamine (GlcNS(3S,6S)) and/or a free amine group (GlcNH₃+) may also be present.

### The Nanoprobe

An exemplary nanoprobe has a general formula [D-G]-M in which D is a fluorescent dye, G is a glycoasminoglycan (GAG) molecule, and M is a metal nanoparticle.

The GAG that is part of the nanoprobe may be any GAG of interest that is capable of being attached, usually covalently, to both a signaling molecule such as a fluorescent dye and a metal nanoparticle. The GAGs generally have the following characteristics: they are susceptible to cleavage by an enzyme of interest; they contain reactive groups capable of being reacted with a metal nanoparticle or a derivative of a metal nanoparticle (e.g. a stabilized or activated metal nanoparticle) and with a reactive group of a detectable dye molecule, so as to form a stable chemical bond, usually a covalent bond; they may contain one or more sulfate groups (—OSO₃⁻) on carbohydrate chains. Exemplary GAGs include but are not limited to heparin, unfractionated heparin, heparin oligosaccharides, low molecular weight heparin, ultra low molecular weight heparin, heparin oligosaccharides, enoxaparin, dalteparin, tinzaparin, fondaparinux, heparan sulfate, dermatan sulfate, chondroitin sulfate, chondroitin sulfate A, chondroitin sulfate B, chondroitin sulfate C, chondroitin sulfate D, chondroitin sulfate E, hyaluronic acid, and keratan sulfate or mixtures thereof.

The signaling molecules and metals that are used in the invention are generally selected as compatible pairs or combinations. The signaling molecules are typically fluorescent dyes, and the dye-metal pairs are selected so that the absorption spectrum of the metal overlaps the emission spectrum of the dye sufficiently to quench fluorescence of the dye, when the two are in close proximity, e.g. within about 200 angstroms of each other, such as when both are attached to a GAG (when the two components are both tethered to a GAG chain).

Exemplary metals that may be used in the practice of the application include but are not limited to: gold, platinum, silver, tungsten and derivatives thereof (such as derivatives produced from pegylation, alkylation, mercaptylation, cellulosylation, etc). For example, gold salt (HAuCl₄) may be reduced to sodium citrate (C₆H₅Na₃O₇-2H₂O) or sodium borohydride (NaBH₄) under surfactant, thereby preparing a stable gold nanoparticle. The diameter of the metal particles used in the nanoprobes of the invention are generally in the size range of from about 5 to about 100 nm, e.g. about 5, 10, 15, 020, 25, 30, 35, 40, 45, 50, 55, 60, 65, 70, 75, 80, 85, 95 or 95 nm in size. The nanometal particles may be of any suitable shape, and are generally roughly spherical with a rough or irregular surface. However, other shapes are not excluded, e.g. strings or clusters of substantially spherical particles, and wires or sheets of nanometer dimensions are also encompassed by the invention.

Exemplary fluorescent dyes that are used include but are not limited to: hylite-594, Alexa Fluor, DyLight, fluorescein, dansyl, cyanine, tetramethylrhodamine, sulforhodamine, HPTS, boron-dipyrromethene (BODIPY) dyes, and other related fluorophores such as eosine derivatives, flavin derivatives, and coumarin derivatives. In exemplary embodiments, the dyes used contain covalent reactive groups, such as hydrazide, amide, ester, ether and others, which include, but are not limited to, hylite-594, Alexa Fluor, and DyLight. Preferably, the fluorophore being used herein emits red or near-infrared fluorescence and has a high quantum yield.

Exemplary fluorescent dyes that may be paired with gold include but are not limited to: fluorescein derivatives, rhodamine derivatives, DyLight® and Alexa Fluor®.

### Coupling of Nanoprobe Components

Coupling of GAG to the metal nanoparticle and/or to the dye molecule is performed through one or more of the following: the reducing end of a GAG chain, the non-reducing end of a GAG chain, via one or more amines, via one or more hydroxyl groups, via one or more carboxylic acid groups and/or via one or more sulfate groups of the GAG.

Dye molecules, especially fluorescent dye molecules, may be selected from existing commercial dyes, or designed or modified to include linking or reactive groups capable of undergoing chemical coupling reactions with reactive groups of interest on other molecules such as GAGs. For example, coupling of the dye to the glycosaminoglycan is generally performed, for example, using one or more alkyl, polyalkyloxy, ester, thioester, and/or other linkers of variable lengths. The length should be sufficient to allow the enzyme to access the GAG chain (i.e. sufficient to prevent steric hindrance) and to maintain a suitable distance between the dye molecule and the metal nanoparticle so that quenching can occur. The length of the linker is generally in the range equating to about 1 to 80 carbons. In general, multiple fluorophore moieties are covalently bound to the GAG tether, thereby increasing the sensitivity of the nanosensor by several orders of magnitude above that of prior art, sensors, which generally rely on fluorophore conjugation to one specific location of a tether. The number of fluorophore moieties present in a single nanoprobe is generally in the range of from a few dozen to several hundred, or even a few thousand. Likewise, the number of fluorophore moieties present on a single glycosaminoglycan chain is generally one to a few dozen.

Within a nanoprobe, the GAG moiety connects or tethers the dye to the metal. As will be understood by those of skill in the art, many GAG preparations are highly heterogeneous and include variable degrees of sulfation, amination, variable chain lengths, etc. Thus, generally multiple GAG chains are attached to a given metal nanoparticle. Further, because multiple reactive groups are present in GAGs, multiple dye molecules are usually attached to each of the GAG chains that are attached to the metal particle. In the nanoprobe, because the GAG chains may be of different lengths, and because reactive groups may be at different positions along the length of any given chain, the dye molecules attached to the GAGs may be positioned at different distances from the metal. The differing distances do not impact the functioning of the nanoprobe, because care is taken to insure that the GAG that is used contains glycan chains that, even though heterogeneous, are less about 200 angstroms in length. This insures that the fluorescence from the dye molecules is adequately quenched, so that a contrasting and readily detectable amount of fluorescence is observed (measured) from the freed fluorophore when the GAG chain is cleaved.

Assay Systems, Kits and Methods of their Use

The assay systems and/or kits of the invention generally include i) the nanoprobe and ii) an enzyme that is capable of cleaving the GAG(s) that are present in the nanoprobe. Suitable enzymes include but are not limited to: various hydrolases and eliminases from any species, either in native or recombinant form, examples of which include glycosaminoglycan hydrolases (GH) such as heparanases and glycosaminoglycan eliminases (GE) such as heparinase (heparitinase or heparin lyase) I, II or III, or chondroitinases A, B, C, or ABC, hyaluronase, hyaluronidase, keratanase, etc. Enzyme-GAG pairings are selected so that the enzyme is capable of cleaving the GAG. The cleaving capability may be specific or selective. For example, in assaying a heparin cleaving enzyme, i.e., heparin lyase or heparanase, the GAG to be used as a tether has to be heparin. Likewise, when assaying a chondroitin sulfate cleaving enzyme, e.g., chondroitinase ABC, the GAG to be used as a tether has to be one of the chondroitin sulfates (e.g., chondroitin sulfate A).

Further, the enzyme that is selected must be inhibited by a contaminant of interest that is suspected of being present in samples of interest that are analyzed. For example, OSCS is a contaminant of commercial heparin. OSCS is also an inhibitor of the GAG-cleaving enzyme combination Heparinase I, II and III, and Heparinase I, II and III are capable of cleaving heparin^{35,36}.

In the nanosensor of the invention, an enzyme that cleaves the tether between the metal nanoparticle and the fluorescent moiety is part of the nanoprobe unit and not part of the sample being analyzed. This is in contrast to prior art nanosensors, which rely on the enzyme being part of the sample that is analyzed. In embodiments of the present invention, to carry out an assay for contamination, the nanoprobe is combined with a sample of interest in the presence of a suitable GAG cleaving enzyme in a suitable reaction vessel, e.g. a well of a 96-well plate. The amount of nanoprobe that is used per individual reaction (e.g. per well) ranges from about 0.1 to about 50 nM (preferably 1 to 10 nM). The amount of enzyme that is used is about 0.01 mg/mL to 10 mg/mL and preferably 0.1 mg/mL. Suitable controls can be provided, e.g. solutions in which the enzyme is absent or inhibited, solutions in which the dye is free in solution and fluorescence is maximized, etc. The desirable range of fluorescence that brackets the two extremes (complete enzyme inhibition and no enzyme inhibition) is dependent on the instrument being used, but the change in fluorescence upon treatment with appropriate enzyme should result in detection of small amounts of enzyme inhibitor(s). For example, for OSCS in heparin, detection of extremely low concentrations, e.g. at a sensitivity of 0.1 ppm and/or femtomolar levels is possible. Thus, when the nanoprobe is incubated with a sample of interest in the presence of a heparinase enzyme, less (or possibly no) fluorescence is detected if an inhibitor of the enzyme is present in the sample, compared to a control sample in which the dye is free to fluoresce.

In kits, the nanoprobe and enzyme may either be provided separately in containers, either in solution or in a dried or desiccated form for reconstitution, or may be pre-mixed e.g. in a suitable buffered medium, and ready to distribute into reaction vessels. Alternatively, one or both of the nanoprobe and enzyme may be provided already distributed in the vessels, e.g. in the wells of a microassay plate. In addition, other arrangements of the assay are also encompassed, including, for example, immobilization of the nanoprobes in the reaction vessel(s), placement of components within capillary tubes for a flow-style assay, etc.

The assays and methods of the invention are typically carried out at room temperature (e.g. about 25° C.), but this is not always the case. In some cases, it may be beneficial to carry out the reactions at higher or lower temperatures, e.g. in the range of from about 10 to about 50° C., e.g. in the cold (to slow the reaction), or heated (to speed the reaction) or at 37° C. (to mimic body temperature), and/or to accommodate the optimal temperature for enzyme activity, or for any other reason.

The assays are generally carried out in an aqueous buffer at a pH that permits adequate enzyme activity, which may be optimum enzyme activity, e.g. typically near neutrality at a pH of about 6.0 to 8.0, e.g. about 6.0, 6.5, 7.0, 7.4, or 8.0.

The assays of the invention are rapid, typically requiring only about 30 minutes of incubation to achieve maximal detection sensitivity. Reactions may be carried out, for example, for periods of time ranging from about 1 minute to about 60 minutes, e.g. for about 1, 5, 10, 15, 20, 25, 30, 35, 40, 45, 50, 55, or 60 minutes, or even longer or shorter, depending on the reaction conditions and the desired assay parameters. The rapidity and sensitivity of the tests means that the assays are well-suited for being adapted to high-throughput screening applications. Further, due to the high sensitivity of the assays, only μg quantities of a sample compound of interest is required to carry out the test.

An example of the practice of the invention is provided in the Examples section below. The example uses nanoparticles comprising heparin thiol, hylite-594 hydrazide, and gold. The nanoprobe was incubated with the GAG eliminase heparitinase in test samples of a heparin solution to detect the presence of contaminants that would inhibit the eliminase activity. In a test sample free of contaminants, the eliminase degraded the heparin on the nanoprobe and the fluorescent dye was released, generating a fluorescent signal. In the presence of the exemplary contaminant OSCS eliminase activity is inhibited, and fluorescence does not increase over background levels measured in blank control reactions.

### APPLICATIONS OF THE INVENTION

The invention provides a simple assay system that can detect contaminants such as OSCS, e.g. at the femtomolar level in a sample of interest. However, applications of this technology are not limited to the analysis of heparin, or to the detection of contaminants. Any sample that might contain a substance of interest that inhibits an enzyme that cleaves or degrades a GAG that can be used as a tether as described herein may be analyzed as described herein.

In some aspects, the samples that are analyzed are glycosaminoglycan samples such as heparin, unfractionated heparin, heparin oligosaccharides, low molecular weight heparin, ultra low molecular weight heparin, enoxaparin, dalteparin, tinzaparin, fondaparinux, dermatan sulfate, chondroitin sulfate, hyaluronic acid or other glycosaminoglycans. The GAGs may be of pharmacologic or non-pharmacologic value. The samples that are tested can be obtained during industrial preparation of glycosaminoglycans, or may be biological fluids such as plasma, blood, urine, saliva, semen, etc.

In other aspects, samples of molecules other than GAGs are assessed for the presence of a molecule of interest. The molecule of interest may be a contaminant (e.g. an unwanted substance), or may be a substance that is wanted but for which the concentration and/or activity is unknown and is to be determined. For example, the levels of various proteins, enzymes, inhibitors, small molecules, drugs, metals, mixtures of polymers, chemically modified GAGs, etc. which have the ability to inhibit a GAG-cleaving enzyme may be measured. Measurements may be carried under in vitro, e.g., the sample arises from a manufacturing or isolation process, or ex vivo conditions, wherein a biological fluid is extracted from an animal and treated soon thereafter for inhibitor or inhibition analysis. Measurements may also be carried out in vivo, wherein an appropriate choice of fluorophore is used to detect the real time release or inhibition of GAG-cleaving enzyme by detecting fluorescence using in vivo fluorescence microscopy.

In some aspects, the assays are employed to determine the level or activity of a protein, such as a coagulation factor. For example, the concentrations and/or activities of proteins such as antithrombin, heparin cofactor II, protease nexin I, protein C inhibitor, and antitrypsin are measured, e.g. using enzymes such as heparanases, heparinases, chondroitinases, sulfotransferases, sulfatases, or coagulation enzymes. Inhibitors that are detected include small molecules that inhibit heparanases, heparinases, chondroitinases, sulfotransferases, sulfatases, or coagulation enzymes. The assays of the invention may also be used, for example, to calibrate the activity of commercial GAG-cleaving or degrading enzymes such as heparitinase, and other enzymes described herein.

In some aspects, the assays are employed to determine the level or activity of a glycan, such as an inhibitor of coagulation. In other aspects, the nanoparticles are used for assessing the levels or therapeutic activity of glycans under in vitro or in vivo conditions. Exemplary glycans that can be assessed include but are not limited to: heparin, unfractionated heparin, heparin oligosaccharides, low molecular weight heparin, enoxaparin, dalteparin, tinzaparin, fondaparinux, dermatan sulfate, chondroitin sulfate, hyaluronic acid or other glycosaminoglycans. In some aspects, the GAG that is detected may be the same as the GAG that serves as the tether, and both may compete for binding to the enzyme such that high levels of GAG in the sample will compete out or at least slow the cleavage of the tethering GAG. Alternatively, the GAG in a sample may differ from that of the tether and inhibit the interaction of the enzyme with the GAG of the tether by another mechanism.

In some aspects, the nanoprobes are used for industrial or forensic applications. For example, a collection of nanoprobes with different types of GAG tethers may be used to detect the presence, nature and concentration of a GAG cleaving enzyme in an unknown sample. Likewise, through competitive processes, the collection of nanoprobes with different types of GAG tethers may be used to detect the presence of competing GAG molecules in an unknown sample.

### EXAMPLES

Here, we report a fast and ultrasensitive analytical assay to detect e.g. 10⁻⁹% w/w OSCS impurities in heparin using a novel Au-Heparin-Dye nano-probe. The studies were conveniently carried out in 96-well microplates using a fluorescence plate reader and were validated by imaging experiments in an in vivo imaging system (IVIS). The nanoprobe provides ultra-sensitive detection of OSCS contamination in heparin, with limits of detection that are far lower than those of the prior art.

### Example 1

### Preparation of Gold-Heparin-Dye Nanoparticle

Gold nanoparticles (Au NPs) exert both fluorescence resonance energy transfer (FRET) and nanometal surface energy transfer (NSET) on fluorophores that are present on or near their surfaces. FRET dictates the quenching property of Au NPs at a separation distance of 100 Å between the dye and the Au NP. NSET takes over beyond 100 Å and maintains the quenching character of Au NP up to a separation distance of 220 Å.^{27,28} We hypothesized that an Au-NP-Hep-Dye conjugate would demonstrate strong fluorescence quenching when the dye is conjugated, and that the fluorescence quenching would be reduced as dye particles were released from the nanoparticle upon enzyme treatment (cleavage) of the heparin linker. A sodium citrate stabilized Au-Hep-Dye nanoparticles using an established protocol. The synthesized conjugate was then tested for its fluorescence quenching properties and subsequently the dye was tested for its fluorescence recovery upon enzyme treatment.

To synthesize Au-Hep-Dye nanosensors, a ligand exchange protocol was followed:
Step 1: Au NPs were synthesized as previously reported.^{31,32} Briefly, 100 mL of 0.5 mM HAuCl₄.3H₂O (0.02 g in 100 mL milli Q water) was heated to 100° C. in an oil bath under vigorous stirring for 30 min. Next, 10 mL of 150 mM sodium citrate dihydrate solution (0.44 g in 10 mL milli Q water) was added into the above solution with continuous boiling. The color of the solution changed to purple in 2-6 min and to ruby red in 6-10 min. The reaction was taken out of oil bath at 8 min and allowed to reach room temperature. The concentration of the Au NP in solution (1.4 nM) was calculated using Beer-Lambert's law. Sodium citrate stabilized Au NPs were stable for two weeks.
Step 2: An aliquot of bleached heparin (1 mg) (from commercial sources, e.g., Sigma) from a 10 mg/mL stock solution was used for chemical thiolation. Heparin (1 mg) was treated with the crosslinker 3-(2-pyridyldithio)propionyl hydrazide (PDPH, 230 μg), EDC (water-soluble carbodiimide crosslinker that activates carboxyl groups for spontaneous reaction with primary amines) (34 μg) in a 50 mM phosphate buffered saline (PBS) buffer, pH 7.4 (1 mL) for 12 h at room temperature. The reaction mixture was, then, purified through ultracentrifugation using molecular weight cut-off (MWCO) filter 3000 at 10000 g for 10 min. The ultracentrifugation process was repeated seven times. The final concentration of heparin-thiol was brought to 10 μL by adding milli Q water.
Step 3: Heparin-thiol (1 mg) was incubated with 5 mM solution of the reducing agent tris(2-carboxyethyl)phosphine) (TCEP, 500 μL) for half an hour to reduce potential disulfide (S—S) bridge formation in the heparin solution. TCEP was removed through ultracentrifugation using MWCO 3000 at 10000 g for 10 min. Heparin-thiol (1 mg) was mixed with EDC (2 mg) and the fluorescent dye hylite-594 hydrazide (330 μg). The coupling reaction was carried out in a 50 mM PBS buffer, pH 7.4 (1 mL) for 16 h at room temperature. Excess dye and EDC were removed through ultracentrifugation using MWCO 3000 at 10000 g for 10 min. The ultracentrifugation process was repeated 10 times.
Step 4: Thiolated heparin-dye conjugate (1 mg) was incubated with 5 mM solution of TCEP (500 μL) for half an hour followed by removal of excess TCEP through ultracentrifugation. Sodium citrate stabilized Au NPs (10 mL, 1.4 nM) were treated with thiolated heparin-dye conjugate (1 mg) under conditions that permitted ligand exchange. The reaction was shaken in an orbital rotor at 37° C. for two days. The heparin-thiol-dye stabilized Au NPs (“AGD NPs”) were then concentrated via ultracentrifugation at 4000 g for 30 min (MWCO 3000).

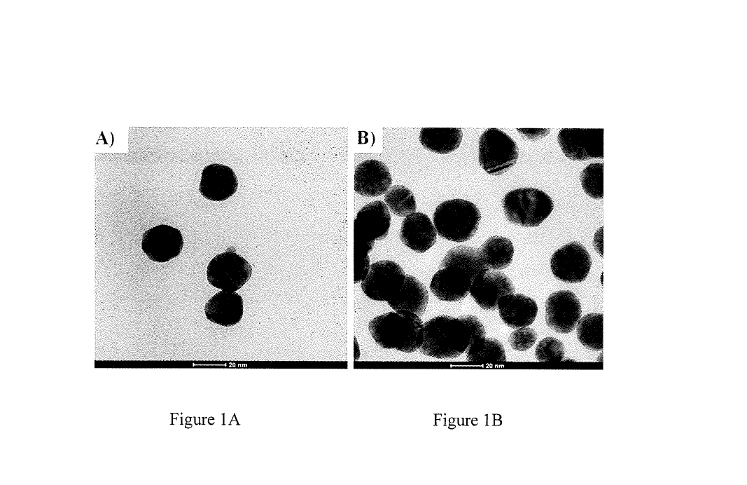

The structural morphology of the resulting Au-heparin-dye nanosensor was observed through transmission electron microscopy before (FIG. 1A) and after (FIG. 1B) exposure to heparitinase I, II, and III. The average particle sizes were 18 nm before and after exposure to the mixture of heparitinase.

### Example 2

### Fluorescence Properties of AGD Nanoparticle

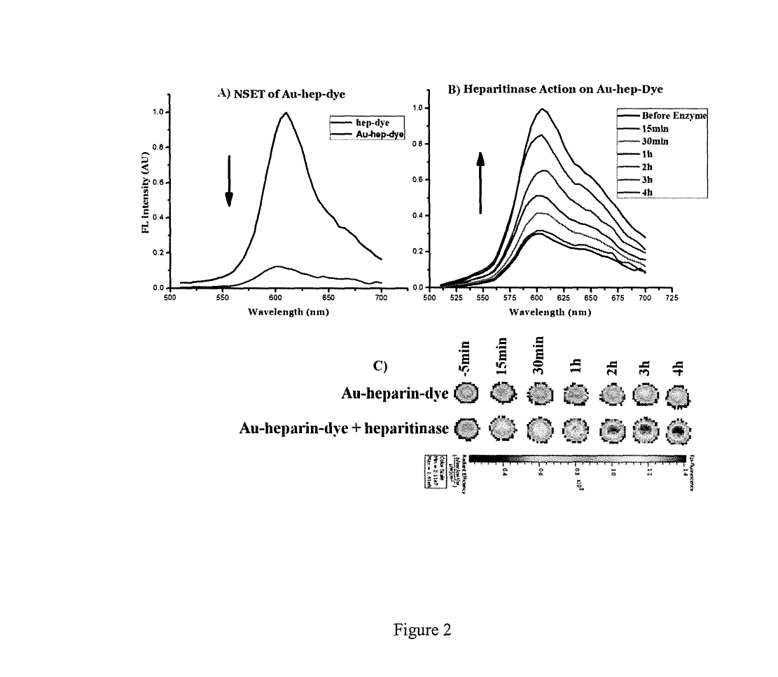

Next, the quenching capability of the nanoparticle conjugates was determined using a fluorescence spectrophotometer. An 88% reduction in fluorescence intensity of the dye was observed after conjugation with the Au NPs, suggesting an efficient energy transfer from the dye to the Au NP. (FIG. 2A).

The Au-heparin-dye nanosensor was then incubated in microplate wells with a mixture of heparitinase enzymes I, II, and III at 37° C. and fluorescence was monitored at various time intervals for up to 4 hours in order to study the efficacy of the enzyme action on the probe. Indeed, a fluorescence recovery of about 70% was observed over a period of 4 h. (FIG. 2B).

The efficacy of heparitinase I, II, III on Au-heparin-dye probe was further tested by incubating the enzyme (10 μL) with the probe (100 0.20 μL) in presence of 10× heparitinase buffer (10 μL) at 37° C. The fluorescence recovery spectra were recorded at incubation times 15 min, 30 min, 1 h, 2 h, 3 h and 4 h. The 96-well plate was excited at 488 nm and a heat map of the 96-well microplate was imaged using an IVIS® imaging system. The results are presented in FIG. 2C. As can be seen, fluorescence increased with increasing incubation time as predicted.

### Example 3

### Study of Inhibition of Heparitinases

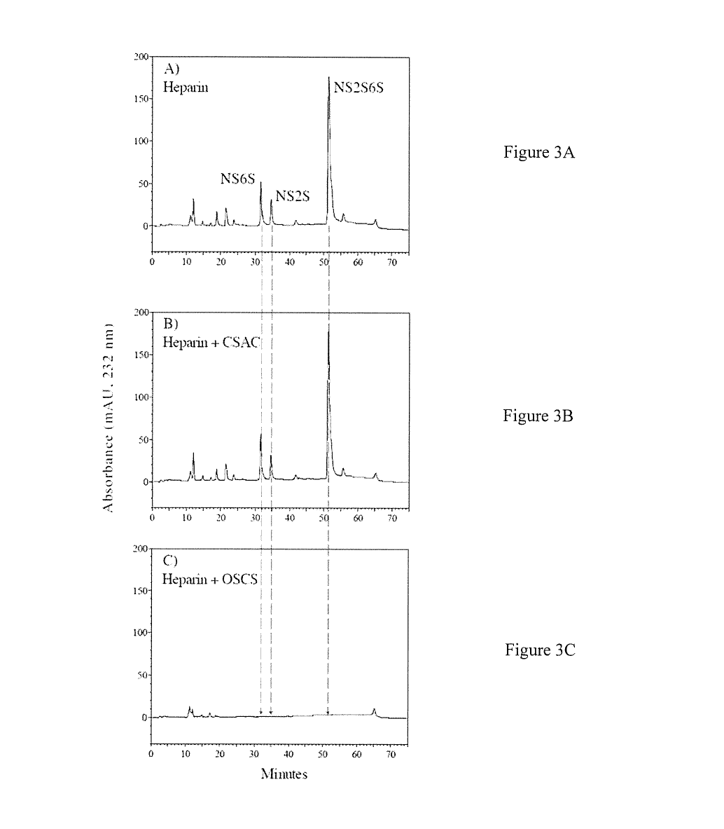

Once it was established that enzyme action causes the fluorescence probe to recover from being quenched, a heparitinase enzyme inhibition study was carried out in the presence of OSCS-contaminated heparin. For this study, a mixture of heparitinase I, II, and III was incubated with (a) heparin (200 μg), (b) heparin with 10% w/w chondroitin sulfate A and C and (c) heparin with 10% w/w OSCS. The resulting oligosaccharides were then analyzed by analytical HPLC. The results showed that 10% w/w OSCS is a powerful inhibitor of heparitinase enzyme activity (FIG. 3A-C). In the presence of 10% w/w OSCS (FIG. 3C), the heparitinases could not act on the Au-heparin-dye nanosensor and fluorescence quenching was maintained. The enzyme activity on the nanosensor should be maximal at low OSCS concentration and minimal at high OSCS concentration. Consequently, the fluorescence quenching of the dye should be minimal at low OSCS concentrations (and measured fluorescence should be high) and quenching should be maximal at high OSCS concentrations (and measured fluorescence should below).

### Example 4

### Diagnostic Kit for Detecting the Presence of Inhibitors

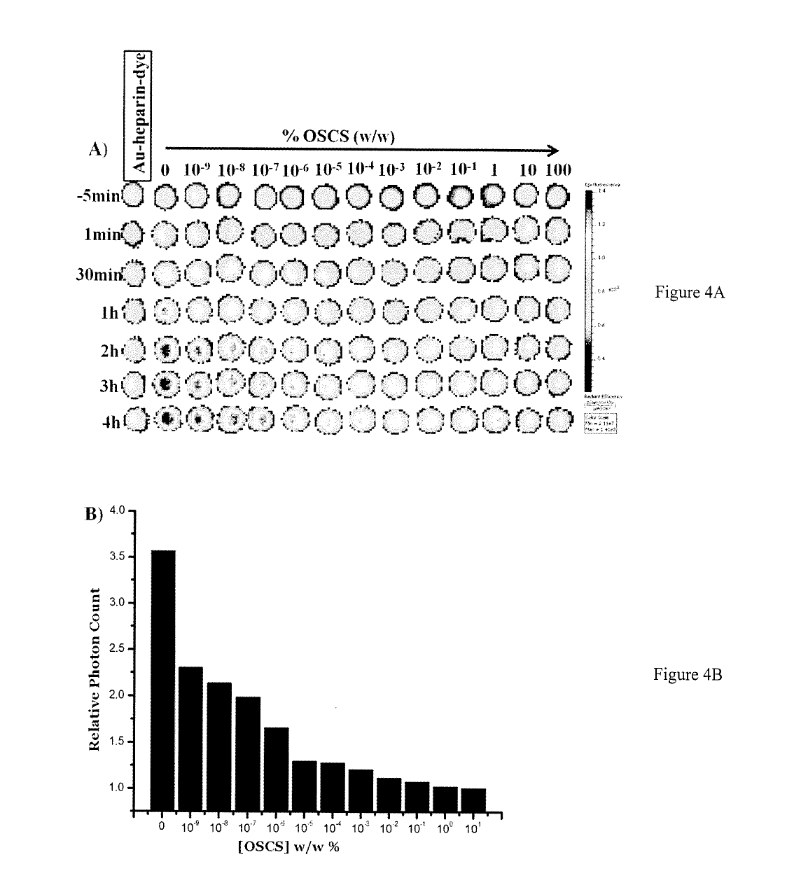

In order to develop an analytical method and kit for determining contamination in heparin, serial dilutions of OSCS were performed at log increment concentrations from 0.1 μg/μL to 0.1 femtogram (fg)/μL. Next, standard heparin solutions (10 μg) were spiked serially with OSCS solutions from 1 μg (10% w/w) to 0.1 fg (10⁻⁹% w/w) of OSCS “contamination” resulting in 11 separate tests. Heparin (10 μg) and OSCS (10 μg) were utilized as positive and negative controls, respectively. When the “contaminated” heparin samples were incubated with the nanosensor described above and with heparitinase, the fluorescence intensity of the dye recovered quickly in samples with limited contamination (FIG. 4A) Surprisingly, differences in the enzyme activity could be observed within 30 minutes of incubation owing to the rapid nature of the assay, and, it was possible to observe differences in enzyme activity even at femtomole concentrations of OSCS-contamination.

An imaging experiment was designed to capture the emitting photons after treating the nano-probe with heparitinase/OSCS spiked heparin. Images were taken after exciting the samples with 535 nm and recording emission using a DSred filter (575 nm-650 nm). The images confirmed the gradient decrease in photons as OSCS contamination increases, even at femtomole concentrations (FIG. 4B).

### Conclusions

Herein, a fast, ultrasensitive nanoprobe and diagnostic kit to detect OSCS contaminant in heparin using Au-heparin-dye nanoprobe is described. The Au-heparin-dye nanoprobe construct was produced using a facile and cost-effective synthetic protocol. Rapid screening of heparin and OSCS contaminated heparin solutions was accomplished within half an hour of incubation with the nanoprobe, and a Limit of Detection (LOD) of 0.1 parts per ten million (0.1 ppm) was achieved. The nanoprobe can thus be used in rapid, high throughput sample screening, for example to determine the quality of heparin in the marketplace before reaching hospitals to maintain a high quality global heparin supply chain and thereby save human lives. This nano probe also has applications in forensic science, for calibration of the activity of commercial lots of heparitinase enzymes, and for monitoring dynamic activities of enzymes in vitro and in vivo during various normal and patho-physiological events.

This is the first ultra-sensitive nanosensor based on a gold nanoparticle to detect OSCS contaminant in heparin that can detect 10⁻⁹% w/w OSCS in heparin. This nano-probe has thus pushed the limit of detection from 0.003% to 10⁻⁹%, a major improvement from the existing colorimetric assay. Detection limits of 0.1 parts per million were achieved using this technology. This represents a significant improvement over the current technology available to detect OSCS, since the present nanosensor requires only μg levels of a compound of interest to detect femtogram quantities of contaminant.

### REFERENCES

1. Dolovich, L. R.; Ginsberg, J. S.; Douketis, J. D.; Holbrook, A. M.; Cheah, G. Arch. Intern. Med. 2000, 160, 181-188
2. Segal, J. B.; Bolger, D. T.; Jenckes, M. W.; Krishnan, J. A.; Streiff, M. B.; Eng, J.; Tamariz, L. J.; Bass, E. B. Am. J. Med. 2003, 115, 298-308
3. Theroux, P. Ouimet, H.; McCans, J.; Latour, J. G.; Joly, P.; Levy, G.; Pelletier, E.; Juneau, M.; Stasiak, J.; deGuise, P.; Guy, B.; Pelletier, G. B.; Ringzler, D.; Waters, D. D. N. Eng. J. Med. 1988, 319, 1105-1111
4. Linhardt, R. J. J. Med. Chem. 2003, 46, 2551-2564
5. Rabenstein, D. L. Nat. Prod. Rep. 2002, 19, 312-331
6. Guerrini, M.; Beccati, D.; Shriver, Z.; Naggi, A.; Viswanathan, K.; Bisio, A.; Capila, I.; Lansing, J. C.; Guglieri, S.; Fraser, B.; Al-Hakim, A.; Gunay, N. S.; Zhang, Z.; Robinson, L.; Buhse, L.; Nasr, M.; Woodcock, J.; Langer, R.; Venkataraman, G.; Linhardt, R. J.; Casu, B.; Torri, G.; Sasisekharan, R. Nat. Biotechnol. 2008, 26, 669-675
7. Kishimoto, T. K.; Viswanathan, K.; Ganguly, T.; Elankumaran, S.; Smith, S.; Pelzer, K.; Lansing, J. C.; Sriranganathan, N.; Zhao, G.; Galcheva-Gargova, Z.; Al-Hakim, A.; Bailey, G. S.; Fraser, B.; Roy, S.; Rogers-Cotrone, T.; Buhse, L.; Whary, M.; Fox, J.; Nasr, M.; Dal Pan, G. J.; Shriver, Z.; Langer, R. S.; Venkataraman, G.; Austen, K. F.; Woodcock, J.; Sasisekharan, R. N. Engl. J. Med. 2008, 358, 2457-2467
8. Acute allergic-type reactions among patients undergoing hemodialysis—multiple states, 2007-2008. Morbid. Mortal. Wkly Rept. 57 (1 Feb. 2008)
9. McMohan, A. W.; Pratt, R. G.; Hammad, T. A.; Kozlowski, S.; Zhou, E.; Lu, S.; Kulick, C. G.; Mallick, T.; Dal Pan, G. Pharmacoepidemiol. Drug Saf. 2010, 19, 921-933
10. US-FDA, Medical devices and diagnostic products that may contain or be coated with heparin, 2009. http://www.fda.gov/MedicalDevices/Safety/AlertsandNotices/ucm135347.htm (accessed Apr. 1, 2013).
11. Mcewen, I.; Mulloy, B.; Hellwig, E.; Kozerski, L.; Beyer, T.; Holzgrabe, U.; Wanko, R.; Spieser, J. M.; Rodomonte, A. Pharmeuropa Bio 2008, 2008, 31-39
12. Trehy, M. L.; Reepmeyer, J. C.; Kolinsky, R. E.; Westenberger, B. J.; Buhse, L. F. J. Pharm. Biomed. Anal. 2010, 49, 670-673
13. Volpi, N.; Maccari, F.; Linhardt, R. J. Anal. Biochem. 2009, 388, 140-145
14. Zhang, Z.; Li, B.; Suwan, J.; Zhang, F.; Wang, Z.; Liu, H.; Mulloy, B.; Linhardt, R. J. J. Pharm. Sci. 2009, 98, 4017-4026
15. Spencer, J. A.; Kauffman, J. F.; Reepmeyer, J. C.; Gryniewicz, C. M.; Ye, W.; Toler, D. Y.; Buhse, L. F.; Westenberger, B. J.; J. Pharm. Sci. 2009, 98, 3540-3547
16. Wang, L.; Buchanan, S.; Meyerhoff, M. E.; Anal. Chem. 2008, 80, 9845-9847
17. Sommer, C. D.; Mans, D. J.; Mecker, L. C.; Keire, D. A. Anal. Chem. 2011, 83, 3422-3430
18. Luhn, S.; Schiemann, S.; Alban, S. Anal. Bioanal. Chem. 2011, 399, 681-690
19. Tami, C.; Puig, M.; Reepmeyer, J. C.; Ye, H.; D'Avignon, D. A.; Buhse, L.; Verthelyi, D. Biomaterials 2008, 29, 4808-4814
20. Ho, H. A.; Boissinot, M.; Bergernon, M. G.; Corbeil, G.; Dore, K.; Boudreau, D.; Leclerc, M. Angew. Chem. Int. Ed. 2002, 41, 1548-1551
21. Lee, K.; Hyukjin, L.; Bae, K. H.; Park, T. G. Biomaterials 2010, 31, 6530-6536
22. Marradi, M.; Alcántara, D.; de la Fuente, J. M.; García-Martín, M. L.; Cerdán S.; Penadés, S. Chem. Commun. 2009, 3922-3924
23. Brinãs, R. P.; Sundgren, A.; Sahoo, P.; Morey, S.; Rittenhouse-Olson, K.; Wilding, G. E.; Deng, W.; Barchi, Jr., J. J. Bioconj. Chem. 2012, 23, 1513-1523
24. Baram-Pinto, D.; Shukla, S.; Aharon Gedanken, A.; Sarid, R. Small, 2010, 6(9), 1044-1050
25. Duchesne, L.; Gentili, D.; Comes-Franchini, M. Fernig, D. G. Langmuir, 2008, 24, 13572-13580
26. Kikkeri, R.; Padler-Karavani, V.; Diaz, S.; Verhagen, A.; Yu, H.; Cao, H.; Langereis, M. A.; De Groot, R. J.; Chen, X.; Varki, A. Anal. Chem. 2013, 85, 3864-3870
27. Yun, C. S.; Javier, A.; Jennings, T.; Fisher, M.; Hira, S.; Peterson, S.; Hopkins, B.; Reich, N. O.; Strouse, G. F. J. Am. Chem. Soc. 2005, 127, 3115-3119
28. Singh, M. P.; Strouse, G. F. J. Am. Chem. Soc. 2010, 132, 9383-9391
29. Labadie, J. Int. J. Risk & Safety in Med. 2012, 24, 37-39
30. Nadanaka, S.; Zhou, S.; Kagiyama, S.; Shoji, N.; Sugahara, K.; Sugihara, S.; Asano, M.; Kitagawa, H. J. Biol. Chem. 2013, 288, 9321-9333
31. Huang, Y.; Kim, D-H.; Langmuir, 2011, 27, 13861-13867
32. Dey, P.; Blakey, I.; Thurecht, K. J.; Fredericks, P. M. Langmuir, 2013, 29, 525-533
33. Nagasawa, K.; Uchiyama, H.; Wajima, N. Carb. Res. 1986, 158, 183-190
34. Maruyama, T.; Toida, T.; Imanari, T.; Yu, G.; Linhardt, R. J. 1998, 306, 35-43
35. Kalita, M.; Balivada, S.; Swarup, V.; Mencio, M.; Raman, K.; Desai, U. R.; Troyer, D.; Kuberan, B.* Development of a nanosensor for fast, ultrasensitive detection of oversulfated chondroitin sulfate contaminant in heparin. Journal of American Chemical Society 2014, 136, 554-557.
36. Desai, U. R.; Wang, H.-M.; Linhardt, R. J.* Specificity studies on the heparin lyases from Flavobacterium heparinum. Biochemistry 1993, 32, 8140-8145.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A nanoprobe for detecting at least one contaminant in a test sample of heparin or a heparin-containing solution, said nanoprobe having a general formula
[D-G]-M
wherein
D is a fluorescent dye,
G is a glycosaminoglycan (GAG), and
M is a metal nanoparticle capable of quenching fluorescence of said fluorescent dye wherein said fluorescent dye is covalently linked to said GAG and said GAG is covalently linked to said metal nanoparticle;
and wherein a distance between said fluorescent dye and said metal nanoparticle in said nanoprobe is such that fluorescence from said fluorescent dye is quenched by said metal nanoparticle.

2. The nanoprobe of claim 1, wherein said fluorescent dye contains a hydrazide reactive group.

3. The nanoprobe of claim 1, wherein said GAG is selected from the group consisting of heparin, unfractionated heparin, a heparin oligosaccharide, a low molecular weight heparin, ultra low molecular weight heparin, enoxaparin, dalteparin, tinzaparin, fondaparinux, dermatan sulfate, chondroitin sulfate and hyaluronic acid, or mixtures thereof.

4. The nanoprobe of claim 1, wherein said metal nanoparticle is selected from the group consisting of gold, platinum, silver, and tungsten.

5. The nanoprobe of claim 4, wherein said metal nanoparticle is gold.

6. The nanoprobe of claim 1, wherein said fluorescent dye is covalently linked to said glycosaminoglycan by a linker selected from the group consisting of alkyl, polyalkyloxy, ester and thioester linkers of variable lengths.

7. The nanoprobe of claim 1, wherein said metal nanoparticle is linked to said glycosaminoglycan at a moiety selected from the group consisting of a reducing end, a non-reducing end, an amine group, a hydroxyl group, a carboxylic group and a sulfate group.

8. The nanoprobe of claim 1, wherein said test sample of heparin or heparin-containing solution is from a pharmaceutical or industrial preparation of a glycosaminoglycan selected from the group consisting of heparin, unfractionated heparin, heparin oligosaccharides, low molecular weight heparin, enoxaparin, dalteparin, tinzaparin, fondaparinux, dermatan sulfate, chondroitin sulfate and hyaluronic acid, or mixtures thereof.

9. The nanoprobe of claim 1, wherein said test sample of heparin or heparin-containing solution is a biological fluid is selected from the group consisting of blood, plasma, serum, urine, saliva, semen, spinal fluid, synovial fluid and ascites fluid.

10. The nanoprobe of claim 1, wherein said metal nanoparticle is gold, said glycosaminoglycan is heparin and said fluorescent dye contains a hydrazide reactive group.

11. The nanoprobe of claim 9, wherein said nanoprobe emits a fluorescent signal higher than a background level in the absence of oversulfated chondroitan sulfate in said test sample of heparin or heparin-containing solution.

12. A method of detecting at least one sulfated polysaccharide contaminant in a heparin preparation or heparin-containing solution, comprising the steps of
I. mixing a test sample of said heparin or a heparin-containing solution with
i) a nanoprobe comprising a fluorescent dye covalently linked to a glycoasminoglycan, which is linked to a metal nanoparticle, having a general formula of
[D-G]-M
wherein
D is a fluorescent dye,
G is a glycoasminoglycan (GAG), and
M is a metal nanoparticle capable of quenching fluorescence of said fluorescent dye
wherein said fluorescent dye is covalently linked to said GAG and said GAG is covalently linked to said metal nanoparticle;
and wherein a distance between said fluorescent dye and said metal nanoparticle in said nanoprobe is such that fluorescence from said fluorescent dye is quenched by said metal nanoparticle, and
ii) a glycosaminoglycan hydrolase or eliminase enzyme;
II. incubating said test sample of said heparin or a heparin-containing solution, said nanoprobe and said glycosaminoglycan hydrolases or eliminase enzyme under suitable conditions to permit activity of said glycoaminoglycan hydrolase or eliminase enzyme,
III. determining the presence or absence of fluorescence with a suitable photo-collector, and
IV. quantitating said fluorescence by comparison with reference values, wherein absence of fluorescence higher than background level is an indication of the presence of said at least one highly sulfated polysaccharide in said test sample of said heparin or a heparin-containing solution.

13. The method of claim 12, wherein said test sample of heparin or heparin-containing solution is from a pharmaceutical or industrial preparation of glycosaminoglycan comprises selected from the group consisting of heparin, unfractionated heparin, heparin oligosaccharides, low molecular weight heparin, enoxaparin, dalteparin, tinzaparin, fondaparinux, dermatan sulfate, chondroitin sulfate and hyaluronic acid, or mixtures thereof.

14. The method of claim 12, wherein said metal nanoparticle is gold, said glycosaminoglycan is heparin and said fluorescent dye contains a hydrazide reactive group.

15. The method of claim 12, wherein said nanoprobe emits a fluorescent signal in the presence of oversulfated chondroitan sulfate in said test sample of heparin or heparin-containing solution.

16. The method of claim 12, wherein said glycosaminoglycan hydrolase or eliminase enzyme is selected from the group consisting of heparanase, heparitinase I, heparitinase II, heparitinase III, (or heparin lyases I, II, III, or heparinases I, II, III), chondroitinase A, chondroitinase B, chondroitinase C, chondroitinase ABC, hyaluronidase, and heparitinase.

17. The method of claim 12, wherein said at least one highly sulfated polysaccharide is oversulfated choindroitin sulfate.

18. The method of claim 12, wherein said glycosaminoglycan hydrolase or eliminase enzyme is a recombinant protein.

19. A method of assessing an amount or therapeutic activity of a protein or glycan present in a patient in need thereof, comprising the steps of
I. mixing a test sample of a biological fluid with
i) a nanoprobe comprising a fluorescent dye covalently linked to a glycoasminoglycan, which is linked to a metal nanoparticle, having a general formula of
[D-G]-M
wherein
D is a fluorescent dye,
G is a glycosaminoglycan (GAG), and
M is a metal nanoparticle capable of quenching fluorescence of said fluorescent dye
wherein said fluorescent dye is covalently linked to said GAG and said GAG is covalently linked to said metal nanoparticle;
and wherein a distance between said fluorescent dye and said metal nanoparticle in said nanoprobe is such that fluorescence from said fluorescent dye is quenched by said metal nanoparticle, and
ii) a glycosaminoglycan hydrolase or eliminase enzyme,
II. incubating said test sample, said nanoprobe and said glycosaminoglycan hydrolases or eliminase enzyme under suitable conditions to permit activity of said glycoaminoglycan hydrolase or eliminase enzyme,
III. determining the presence or absence of fluorescence with a suitable photo-collector, and
IV. quantitating said fluorescence by comparison with a reference value to determine said amount or therapeutic activity of said protein or glycan present in said patient.

20. The method of claim 19, wherein said protein is selected from the group consisting of antithrombin, heparin cofactor II, protease nexin I, protein C inhibitor and antitrypsin a heparanase, a heparinase, a heparitinase, a chondroitinase, a sulfotransferase, a sulfatase and a coagulation enzyme.

21. The method of claim 19, wherein said glycan is selected from the group consisting of heparin, unfractionated heparin, heparin oligosaccharides, low molecular weight heparin, enoxaparin, dalteparin, tinzaparin, fondaparinux, dermatan sulfate, chondroitin sulfate and hyaluronic acid.

22. The method of claim 19, wherein said biological fluid is selected from the group consisting of blood, plasma, serum, urine, saliva, semen, spinal fluid, synovial fluid and ascites fluid.

23. The method of claim 19, wherein said metal nanoparticle is gold, said glycosaminoglycan is heparin and said fluorescent dye contains a hydrazide reactive group.

24. The method of claim 19, wherein said glycosaminoglycan hydrolase or eliminase enzyme is selected from the group consisting of heparanase I, heparanase II, heparanase III, heparain lyase, chondroitinase A, chondroitinase B, chondroitinase C, chondroitinase ABC, hyaluronidase, and heparitinase.

25. The method of claim 19, wherein said glycosaminoglycan hydrolase or eliminase enzyme is a recombinant protein.

26. The method of claim 19, wherein said protein is selected from the group consisting of antithrombin, heparin cofactor II, protease nexin I, protein C inhibitor and antitrypsin a heparanase, a heparinase, a chondroitinase, a sulfotransferase, a sulfatase and a coagulation enzyme.

27. The method of claim 19, wherein said glycan is selected from the group consisting of heparin, unfractionated heparin, heparin oligosaccharides, low molecular weight heparin, enoxaparin, dalteparin, tinzaparin, fondaparinux, dermatan sulfate, chondroitin sulfate and hyaluronic acid, or mixtures thereof.

28. A method of assessing an amount or activity of a glycosaminoglycan (GAG)-cleaving enzyme in a sample of interest, comprising the steps of
I. mixing said sample of interest with a nanoprobe having a general formula
[D-G]-M
wherein
D is a fluorescent dye, G is a GAG that is cleavable by said GAG-cleaving enzyme, and M is a metal nanoparticle capable of quenching fluorescence of said fluorescent dye;
wherein said fluorescent dye is covalently linked to said GAG and said GAG is covalently linked to said metal nanoparticle,
and wherein a distance between said fluorescent dye and said metal nanoparticle in said nanoprobe is such that fluorescence from said fluorescent dye is quenched by said metal nanoparticle;
II. incubating said sample of interest and said nanoprobe under conditions which permit cleavage of said GAG by said GAG-cleaving enzyme;
III. determining at least one level of fluorescence produced in said step of incubating, and
IV. quantitating said at least one level of fluorescence by comparison with at least one reference value to determine said amount or activity of said GAG-cleaving enzyme.

29. The method of claim 28, wherein said method is carried out in the presence of one or more molecules of interest.

30. The method of claim 29, wherein said one or more molecules of interest is/are selected from the group consisting of a molecule that is a known or suspected inhibitor of said GAG-cleaving enzyme, and a molecule that is a known or suspected activator of said GAG-cleaving enzyme.
